(19) United States
(12) Patent Application Publication
(10) Pub. No.: US 2004/0210081 A1
(43) Pub. Date: Oct. 21, 2004

(54) Carboxylic esters based on 2-hydroxymethylnorbornane

(76) Inventors: Peter Lappe, Dinslaken (DE); Helmut Springer, Dinslaken (DE)

Correspondence Address: Charles A. Muserlian, Muserlian, Lucas and Mercanti, 475 Park Avenue South, New York, NY, 10016, US

(21) Appl. No.: 10/804,727

(22) Filed: Mar. 19, 2004

(30) Foreign Application Priority Data
Apr. 15, 2003 (DE) 103 17 204.1

(51) Int. Cl. C07C 69/34; C07C 69/52

(52) U.S. Cl. 560/194

(57) ABSTRACT

The present invention relates to carboxylic esters based on 2-hydroxymethylnorbornane and on aliphatic dicarboxylic acids of the formula

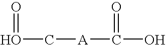

where A is —(CH₂)ₓ— and where x=from 1 to 10, and to a process for their preparation, and also to their use as lubricants or plasticizers for thermoplastics.

The present invention relates to novel carboxylic esters based on 2-hydroxymethylnorbornane, to a process for their preparation, and also to their use.

The industrial uses of carboxylic esters are wide-ranging and varied, examples being plasticizers, lubricants, and fragrances. A large number of different esters are used industrially, extending from simple carboxylic esters composed of monocarboxylic acids and of monoalcohols through complex ester oils composed of mixtures of mono- and dicarboxylic acids with mono- and polyhydric alcohols. The selection of suitable starting products permits controlled adjustment of the physical properties of the material, e.g. boiling point or viscosity, and permits consideration to be given to chemical properties, e.g. hydrolysis resistance or resistance to oxidative degradation. Carboxylic esters can also be tailored specifically to solve particular problems in applications technology.

By way of example, comprehensive overviews of the use of carboxylic esters are found in Ullmann's Encyclopedia of Industrial Chemistry, 5th, edition, 1988, VCH; Vol. A11, pp. 191-193, Vol. A15, pp. 438-440, Vol. A20, pp. 439-458, Common Fragrance and Flavor Materials, Wiley-VCH 2001.

The use of carboxylic esters as lubricants is of great industrial importance. The term “lubricants” encompasses in its strict sense only products which are used for the lubrication of sliding or rolling elements. The lubricants used in numerous applications within industry are mainly composed of mineral oils or of products which are entirely or to some extent synthetic. Lubricants based on mineral oils are highly versatile. They are used not only for lubrication and power transmission at high and low temperatures but also for heat-transfer and insulation. Where requirements cannot be fully met by the mineral oil products, synthetic liquids which resemble lubricating oil can provide solutions with a technical advantage. Synthetic base oils are prepared from substantially homogeneous substances under controlled conditions, and may belong to a variety of classes of chemical compound.

The ester oils represent a particularly important class of compound, and are extensively used, for example in aircraft as turbine engine oils and instrument oils, and as greases or weapons-grade oils. These ester oils are prepared via the reaction of acids or acid anhydrides, in particular of mono- or dicarboxylic acids, with alcohols, in particular mono-, di-, tri- or tetraols.

For acids, examples of industrially important starting materials are aliphatic monocarboxylic acids having from 5 to 10 carbon atoms. Examples of dicarboxylic acids available in industrial quantities are adipic acid, azeleic acid, and sebacid acid. Alcohols which may used, besides the aliphatic alcohols, such as 2-ethylhexanol, are particularly polyhydric alcohols, such as ethylene glycol and its oligomers di-, tri- and tetraethylene glycol, propylene glycol and its oligomers, 1,3-propanediol, 1,4-butanediol, 1,6-hexanediol, neopentyl glycol, trimethylolpropane, glycerol, and pentaerythritol.

The development of modern lubricants and their correct use are of considerable economic significance. Lubricants ideally matched to the respective task yield savings via energy savings, reduced abrasion, reduced maintenance times, and longer periods between overhaul. This means that, although there are already numerous products in everyday use and in industrial use, there is a need for novel lubricants with improved properties.

The use of carboxylic esters as plasticizers is likewise of great economic importance. Plasticizers are widely used in many applications in plastics, coating compositions, sealing compounds, and rubber products. They interact physically with high-molecular-weight thermoplastic polymers, without reacting chemically, and preferably via their capability to solvate and swell. The result is a homogeneous system whose thermoplastic region has been shifted to lower temperatures when comparison is made with the original polymer, one of the results being that its mechanical properties are optimized, for example its deformation capability, elasticity, or strength are increased, and its hardness is reduced. Plasticizers have to comply with a series of criteria if they are to gain access to the widest possible field of applications. Ideally, they should be odorless and colorless, and be resistant to light, low temperatures, and heat. In addition, they are expected to be water-resistant, and to have low combustibility and low volatility, and not to cause any health hazard. In addition, the intention is that the plasticizers be easy to prepare, while avoiding the production of waste materials, such as non-recyclable by-products and polluted wastewater, the intention here being to comply with environmental requirements.

The esters of di- and polycarboxylic acids with plasticizer alcohols, i.e. unbranched or branched primary alcohols having from about 6 to 20 carbon atoms, are among the most important plasticizers, and are used in the form of individual compounds or else mixtures. The esters of adipic acid, of azeleic acid, and of sebacic acid are in particular used as ester plasticizers for plasticizing PVC.

One specific class of ester plasticizers, also known by the abbreviated term G esters, contains, as alcohol component, diols or ether diols, namely ethylene glycol, diethylene glycol, triethylene glycol, and tetraethylene glycol.

As is the case with the lubricants above, the development of modern plasticizers tailored to solve a particular applications problem is of considerable economic significance. Although there are already numerous products in the market, there remains a high level of interest in the need for plasticizers which have better properties and are ideally matched to the respective task.

It is therefore an object of the invention to provide carboxylic esters which can be used particularly successfully as lubricants or as plasticizers. The invention is also based on the provision of a process which permits the preparation of these carboxylic esters from readily accessible starting materials available in adequate quantity at low cost. In this context, it is particularly useful that the esterification process be capable of realization using simple technical means, and requiring no complicated or specialized apparatus.

The present invention provides carboxylic esters of the formula

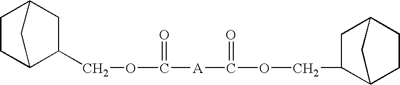

where A is —(CH₂)ₓ—, where x=from 1 to 10.

The preparation of 2-hydroxymethylnorbornane, as alcohol component, takes place via hydroformylation of norbornene, an olefin available in large quantities at low cost, and used industrially, inter alia, for the production of cycloolefin copolymers. The hydroformylation of norbornene with a Rh catalyst gives very high yields of 2-formylnorbornane. The reaction with synthesis gas generally takes place in a homogeneous phase in a conventional organic solvent, such as cyclohexane, toluene, or n-hexane, at temperatures of from 80 to 150° C. and at pressures of 10 to 30 MPa, in the presence of known organic phosphorus(III) compounds, e.g. triphenylphosphine, as ligand. 2-Formylnorbornane is obtained from the crude hydroformylation product via distillative work-up. 2-Formylnorbornane is then reacted with hydrogen at an elevated pressure and an elevated temperature, in the presence of conventional hydrogenation catalysts, to give 2-hydroxymethylnorbornane. Use may be made of the hydrogenation catalysts commonly used in industry, e.g. supported or unsupported metal catalysts comprising, by way of example, nickel, palladium, or copper as catalytically active metal. Promoters, such as zirconium or manganese, may also be present, where appropriate. Conventional support materials are silicon dioxide or aluminum oxide.

The hydrogenation reaction is carried out under conventional temperature conditions in the range from 70 to 150° C. and under conventional pressure conditions in the range from 2 to 30 MPa. The hydrogenation reaction proceeds with high yields. This means that 2-hydroxymethylnorbornane is available at low cost from a technologically simple process for the preparation of novel carboxylic esters.

Dicarboxylic acids used are especially the aliphatic members of the group—malonic acid (x=1), succinic acid (x=2), glutaric acid (x=3), adipic acid (x=4), azeleic acid (x=7), sebacic acid (x=8), and 1,12-dodecanedioic acid (x=10). These simple members of the group of aliphatic dicarboxylic acids are available on an industrial scale, or may be prepared by known processes.

The direct esterification of alcohols with carboxylic acids is one of the fundamental operations of organic chemistry. The reaction is usually carried out in the presence of catalysts in order to increase the reaction rate. The use of one of the reactants in excess, and/or the removal of the water formed during the course of the reaction ensures that the equilibrium is shifted, as required by the law of mass action, towards the side of the reaction product, i.e. of the ester, i.e. that high yields are achieved.

Various processes are known for removing the water of reaction formed during ester formation. Use is preferably made of azeotropic distillation in the presence of a solvent immiscible with water, the heating of the reaction mixture while flushing with an inert gas, or reaction of the alcohol and carboxylic acid starting materials in vacuo, or in the presence of a drying agent.

The removal of water via azeotropic distillation has in particular proven successful for adjusting the equilibrium during the preparation of ester plasticizers. The azeotrope-forming material usually used comprises organic solvents which are available industrially at low cost. However, any of the other organic substances which have an appropriate boiling point and which form an azeotrope with water are suitable. Examples of entrainers used are hexane, 1-hexene, cyclohexane, toluene, and benzene.

The required amount of entrainer for complete removal of the water may be determined in a simple manner from the amount of water formed, calculated from the stoichiometry of the esterification reaction, and the composition of the binary azeotrope. Use of an excess of the entrainer has proven successful, and it is advantageous for the portion of entrainer used to be from 50 to 200% by weight greater than the theoretically calculated amount. The progress of the reaction may be followed in a simple manner via collection and separation of the entrainer/water mixture removed by distillation. The entrainer separated from the azeotrope may be returned directly into the reaction, i.e. without any intermediate purification stage.

The reaction of 2-hydroxymethylnorbornane and carboxylic acid may be carried out without use of catalyst. This version of the reaction has the advantage that no foreign substances which can lead to undesired contamination of the ester are introduced into the reaction mixture. However, the reaction temperatures which have to be maintained are then generally higher, because that is the only way of ensuring that the reaction proceeds at an adequate, i.e. cost-effective, rate. In this context, it should be noted that the raising of the reaction temperature can lead to thermal degradation of the ester. It is therefore not always possible to avoid the use of a catalyst which facilitates the reaction and increases the reaction rate. The catalyst may often be an excess of the acid which is simultaneously a component reacting with the 2-hydroxymethylnorbornane. The other esterification catalysts which affect reaction rate are also suitable, examples being mineral acids, such as sulfuric acid, phosphoric acid, polyphosphoric acid, or acidic salts thereof, trialkyl phosphates or triaryl phosphates, formic acid, methanesulfonic acid, or p-toluenesulfonic acid.

The amount of the catalyst used may vary over a wide range. Use may be made of either 0.01% by weight or else 5% by weight of catalyst, based on the reaction mixture. However, because few advantages result from greater amounts of catalyst, the catalyst concentration is usually from 0.01 to 1.0% by weight, preferably from 0.01 to 0.5% by weight, based in each case on the reaction mixture. For each individual case, preliminary experiments are advantageously used, where appropriate, in order to decide whether operations are to be carried out without catalyst at a relatively high temperature or with catalyst at a relatively low temperature.

The esterification may be used in stoichiometric amounts of 2-hydroxymethylnorbornane and acid. However, it is preferable to use an excess of 2-hydroxymethylnorbornane in order to achieve maximum completion of conversion within a finite time.

The reaction between 2-hydroxymethylnorbornane and the acid begins to occur in the range from about 80 to 140° C., depending on the starting materials. It may be completed at temperatures of up to about 200° C. These temperatures are guideline values which are advantageously maintained. By way of example, lower temperatures may be sufficient if in a specific case an adequately high reaction rate is achieved or only partial conversions are desired. Higher temperatures are possible if it is possible to exclude the appearance of decomposition products which, inter alia, adversely affect color. The use of reduced or increased pressure has not been excluded, but will be limited to special cases.

Once the reaction has ended, the resultant reaction mixture comprises not only the ester, as desired reaction product, but also any unreacted starting materials, and in particular still comprises excess 2-hydroxymethylnorbornane, if operations used an excess of alcohol. For work-up, the reactor discharge is freed from catalyst by conventional methods. If the catalyst takes the form of a solid, e.g. a hydrogensulfate, the product is filtered in conventional filter apparatus at normal temperature or at temperatures up to 150° C. The filtration may be promoted by using familiar filtration aids, such as cellulose, silica gel, kieselguhr, or wood flour. Excess and unreacted starting materials are then removed by distillation. In order to remove final residues of acidic constituents, the mixture may also be treated with an alkaline reagent, e.g. aqueous soda solution or aqueous sodium hydroxide solution. After phase separation, the crude product is subjected to fractional distillation. If the catalyst is present in solution in the reaction mixture, as is the case with sulfuric acid or para-toluenesulfonic acid, any remaining starting materials present are first removed by distillation, where appropriate after previous filtration, and the mixture is then treated with an alkaline reagent, and the crude ester is fractionated in vacuo.

In another version of the work-up, after alkali treatment and phase separation the ester is dried, for example by passing an inert gas through the product, applying vacuum, or adding a solid drying agent, e.g. sodium sulfate or magnesium sulfate, which is removed by filtration once the drying process is complete. Where appropriate, the product is subjected to steam distillation prior to the final drying step.

If the intended use requires this, the isolation of the ester may also be followed by other purification steps, for example a fractional distillation in vacuo.

The esterification reaction may be carried out batchwise or else continuously, in the reaction apparatus typically used in the chemical industry. Apparatus which has proven successful is a stirred tank equipped with a heating device and with a device for introducing the azeotrope-forming material.

The inventive esters have excellent suitability as plasticizers for any of the familiar high-molecular-weight thermoplastic polymers. They may also be used as lubricants, with excellent results.

The examples below serve to illustrate the invention, but the invention is not limited to the examples.

### EXAMPLE 1

Preparation of di(norborn-2-ylmethyl) malonate

82.2 g (0.79 mol) of malonic acid, 218.3 g (1.73 mol) of 2-hydroxymethylnorbornane, 4.2 g (0.022 mol) of p-toluenesulfonic acid, and 50 g of toluene form an initial charge in a 1 l three-necked flask, with stirrer, internal thermometer, and water separator, and are heated to reflux. Within a period of 2 hours, 27.4 g of water are removed from circulation. The reaction mixture is cooled to room temperature and treated with 107.9 g of aqueous sodium hydroxide solution (1% strength). The amount of organic phase remaining after phase separation is 311.9 g, and this is then washed with 239.3 g of water. After a further phase separation, the organic phase (306.4 g) is subjected to fractional distillation. The ester (233.3 g) is isolated at 96.3% purity at a top-of-column temperature of 174° C. and at a pressure of 100 Pa. This corresponds to a yield of 88.8% of theory.

### EXAMPLE 2

Preparation of di(norborn-2-ylmethyl) succinate

As in example 1, 93.3 g (0.79 mol) of succinic acid, 218.3 g (1.73 mol) of 2-hydroxymethylnorbornane, 4.2 g (0.022 mol) of p-toluenesulfonic acid, and 50 g of toluene are reacted. Within a period of 2 hours, 29.1 g of water are removed from circulation. The reaction mixture is cooled to room temperature and treated with 161.7 g of aqueous sodium hydroxide solution (1% strength). The amount of organic phase remaining after phase separation is 321.0 g, and this is then washed with 243.3 g of water. After a further phase separation, the organic phase (291.7 g) is subjected to fractional distillation. The ester (237.5 g) is isolated at 98.1% purity at a top-of-column temperature of 187° C. and at a pressure of 100 Pa. This corresponds to a yield of 88.2% of theory.

### EXAMPLE 3

Preparation of di(norborn-2-ylmethyl) glutarate

As in example 1, 104.3 g (0.79 mol) of glutaric acid, 218.3 g (1.73 mol) of 2-hydroxymethylnorbornane, 4.2 g (0.022 mol) of p-toluenesulfonic acid, and 50 g of toluene are reacted. Within a period of 2 hours, 27.6 g of water are removed from circulation. The reaction mixture is cooled to room temperature and treated with 96.7 g of aqueous sodium hydroxide solution (1% strength). The amount of organic phase remaining after phase separation is 358.0 g, and this is then washed with 241.0 g of water. After a further phase separation, the organic phase (338.0 g) is subjected to fractional distillation. The ester (241.6 g) is isolated at 98.7% purity at a top-of-column temperature of from 184 to 186° C. and at a pressure of 100 Pa. This corresponds to a yield of 86.6% of theory.

### EXAMPLE 4

Preparation of di(norborn-2-ylmethyl) adipate

As in example 1, 115.4 g (0.79 mol) of adipic acid, 218.3 g (1.73 mol) of 2-hydroxymethylnorbornane, 4.2 g (0.022 mol) of p-toluenesulfonic acid, and 50 g of toluene are reacted. Within a period of 2 hours, 28.2 g of water are removed from circulation. The reaction mixture is cooled to room temperature and treated with 88.3 g of aqueous sodium hydroxide solution (1% strength). The amount of organic phase remaining after phase separation is 353.8 g, and this is then washed with 241.7 g of water. After a further phase separation, the organic phase (366.8 g) is subjected to fractional distillation. The ester (247.6 g) is isolated at 98.9% purity at a top-of-column temperature of from 184 to 186° C. and at a pressure of 100 Pa. This corresponds to a yield of 85.5% of theory.

### EXAMPLE 5

Preparation of di(norborn-2-ylmethyl) sebacate

As in example 1, 159.8 g (0.79 mol) of sebacic acid, 218.3 g (1.73 mol) of 2-hydroxymethylnorbornane, 4.2 g (0.022 mol) of p-toluenesulfonic acid, and 50 g of toluene are reacted. Within a period of 2 hours, 29.1 g of water are removed from circulation. The reaction mixture is cooled to room temperature and treated with 13.5 g of aqueous sodium hydroxide solution (10% strength). The amount of organic phase remaining after phase separation is 404.7 g, and this is then washed three times with a total of 682.0 g of water. After a further phase separation, the organic phase (381.8 g) is subjected to fractional distillation. The ester (281.2 g) is isolated at 98.9% purity at a top-of-column temperature of from 220 to 222° C. and at a pressure of 100 Pa. This corresponds to a yield of 84.1% of theory.

1 A carboxylic ester of the formula 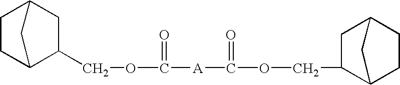
where A is —(CH₂)ₓ—, where x=from 1 to 10.

2 The carboxylic ester as claimed in claim 1, wherein x is 1, 2, 3, 4, 7, 8, or 10.

3 A process for preparing the carboxylic esters as claimed in claim 1 via reaction of 2-hydroxymethylnorbornane with dicarboxylic acids of formula 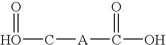
or with their anhydrides of the formula 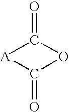
where A is as defined in claim 1, in the presence of an entrainer for removal, in the form of an azeotropic mixture, of the water formed during the course of the reaction, and, where appropriate, in the presence of a catalyst, removal of excess and unconverted starting materials, treatment with an alkaline reagent for removal of acidic constituents, and then, where appropriate, steam distillation, followed by drying and/or fractional distillation.

4 (cancelled)

5 In lubricants or plasticizers provided with thermoplastics, the improvement comprising using the lubricant or plasticizer a compound of claim 1.

6 The thermoplastics of claim 5 wherein x is 1, 2, 3, 4, 7, 8 or 10.
